(19) United States
(12) Patent Application Publication
(10) Pub. No.: US 2015/0065869 A1
(43) Pub. Date: Mar. 5, 2015

(54) LOW NOISE TRANSMISSION SCAN SIMULTANEOUS WITH POSITRON EMISSION TOMOGRAPHY

(71) Applicant: Prescient Imaging LLC, Hawthorne, CA (US)

(72) Inventors: Farhad Daghighian, Hawthorne, CA (US); Yibao Wu, Cypress, CA (US)

(21) Appl. No.: 14/476,634

(22) Filed: Sep. 3, 2014

Related U.S. Application Data
(60) Provisional application No. 61/873,297, filed on Sep. 3, 2013.

(51) Int. Cl. A61B 6/03; G01T 1/29; A61B 6/00

(52) U.S. Cl. CPC A61B 6/037; A61B 6/5205; A61B 6/4266; G01T 1/2985
USPC 600/425

(57) ABSTRACT

A method and device is described for correcting PET images for the attenuation of effects of the tissues and periodic movements of the patient. The device provides gamma-rays for image registration along with the CT scan. The gamma-rays are detected by the same detectors that form the emission PET scan. The device for generating the gamma-rays is a rod that contains an array of detectors, and a radioactive source that emits two rays in coincidence with each other. One of these rays is a gamma ray, and the other can be a beta ray, an alpha ray, or a low energy x-ray. The position of the rod, which is moveable within the ring of the PET scanner, is monitored so that its position can be coordinated with the signals generated from the ring detectors.

### CROSS REFERENCE TO RELATED APPLICATIONS

This application claims the benefit of U.S. Provisional Application Ser. No. 61/873,297 to Farhad Daghighian, entitled TRANSMISSION X-RAY OR GAMMA-RAY COMPUTED TOMOGRAPHY USING A SPECIAL RADIOACTIVE SOURCE THAT IS COUPLED TO A SET OF DETECTORS THAT PROVIDES COINCIDENCE TRIGGERING, filed on Sep. 3, 2013, which is incorporated herein in its entirety by reference, including the drawings, charts, schematics, diagrams and related written description.

### BACKGROUND OF THE INVENTION

1. Field of the Invention

Described herein are devices and methods relating to positron emission tomography (PET) scanning, particularly a PET scanner with improved attenuation correction features and methods for its use.

2. Description of the Related Art

Positron emission tomography (PET) is a commonly-used nuclear medicine, functional imaging technique that produces a three-dimensional image of functional processes in the body. It is an important part of detection and management of cancer, heart disease and brain abnormalities. The system detects pairs of photons emitted indirectly by a positron-emitting radionuclide (tracer), which is introduced into the body on a biologically active molecule, for example, a Fluoride-18 fluorodeoxy-glucose injected intravenously. Three-dimensional images of tracer concentration within the body are then constructed by computer analysis of this emission data set. However, the attenuating effect of the tissues on the emission pattern must be taken into account in order to have an artifact-free and quantitative image of the radioactive distribution. This task is achieved by a “transmission scan”. Most PET scanners today have a CT scanner attached to them and the CT scan of each slice is used to estimate the attenuation of 511 keV photons of the PET emission scan of the same slice of the body.

Before the advent of PET/CT, a rotating radioactive rod was used for transmission scan. In the early models of PET scanners, a rod containing Germanium-68 (Ge-68) was placed inside the detector ring, parallel to the central axis and close to the inner wall of the detector ring. Before the patient is injected with a radioactive tracer, the patient is positioned along the central axis of the detector ring and the rod is rotated around the patient while the angular position of the rod is registered at various stages of the rotation. Ge-68 emits a positron that decays to two back-to-back annihilation photons, each with 511 keV energy emitted simultaneously. An example of this configuration is shown in FIG. 1, which shows the detector-ring 100 of a PET scanner, comprising multiple scanner detectors 102, and a rod 104, containing GE-68. As an emitted gamma ray 106 enters a detector 102 of the PET scanner 100, another annihilation gamma ray 108 traverses the patient's body 110 and is detected by another region of the PET detector 102. The annihilation gamma ray 108 may be absorbed in the body depending on the attenuation of the tissues in the path of this gamma ray which is approximately 511 keV. The rod rotates around the inner periphery of the PET detector-ring 100 at least one complete cycle, and after a few minutes a set of detector responses are collected, and by comparing this data set with a control that was acquired when there was no patient in the scanner (i.e., a “blank scan”), the attenuation images will be formed. Subsequently, while the patient is in the same position, a positron-emitting radioisotope is injected into the patient and a positron emission scan is formed over a period of a few minutes (the “raw emission image”). The attenuation image is then used to correct the raw emission image and form the “attenuation-corrected PET image”, that is accurate and artifact-free.

Some shortcomings of the above-mentioned attenuation correction method include that such method take twice as much time, as one scan must be completed for a transmission image, and one scan for a PET emission scan. It is not possible to do a transmission scan while the patient is injected with a radiation source because there is no way to know if the detected 511 keV gamma rays was from the radioactivity in the rod or from the radioactivity in the patient.

Another conventional way to correct for such attenuation, which is utilized in virtually all current commercially available PET scanners, is to utilize an x-ray computed tomography (CT) scanner in a process known as PET-CT scanning. PET-CT scanning results in images acquired from both devices taken sequentially, in the same session, and combined into a single superposed (co-registered) image. The x-ray CT scanner performs two major functions: 1) It provides an attenuation map for attenuation correction of the PET images; and 2) It provides anatomical images that can be co-registered with PET images thereby helping clinicians to identify the precise location of PET-identified hot spots.

The additional use of CT scanner for attenuation correction increases overall time of scanning, expenses and requires a larger space and due to its increased weight the facilities may need structural reinforcement.

An additional problem with convention PET scanning devices is that the internal organs of the body move due to the rhythmic cycles of the heart or respiration, causing blurring and degradation of resolution and the potential introduction of motion artifacts appearing in the image. The conventional method is called “gating” and typically collects the emission data into different files, with each file corresponding to one time segment of a physiologic cycle, such as the cardiac or respiratory cycle.

In the case of cardiac cycling, the signal from an electrocardiograph lead is continuously acquired during the PET emission scan, and is used to send an electronic signal into the PET scanner to “gate” the emission data into different files. For example all PET detected events during the systolic part of the cardiac cycle are collected in one file, and all the events during the diastolic part of the cardiac rhythm is collected in another file. Similarly, the motion from respiration is transformed to an electronic signal in various ways, such as mechanically using a belt that moves up and down, optically by optical position sensing of marker on the patient's surface, or by monitoring the direction of air flow around the nose and mouth of the patient.

Having these electronic signals enable the PET's data acquisition system to divide the PET data into frames based on various stages of this motion, such as inhale or exhale or in-between positions, typically 4-8 frames. Assuming no or little motion inside one frame, an image of each frame is reconstructed having less motion artifact or blurring.

Since the attenuation correction in PET-CT scanners is done using the CT scan, one has to use the same attenuation map for different frames of the emission scan. Because the position of various organs have changed from frame to frame in the emission images due to cardiac motion or respiratory motion, and is not the same as those when the CT scan was taken, artifacts are introduced in the attenuation corrected images.

Therefore, there is a need for a low-cost mechanism for simultaneous emission and transmission scan to improve upon attenuation correction. Also, there is a need to do transmission scans that are gated the same way as the emission scan following the cardiac and respiratory motions particularly, for example, for accurate imaging of lung cancer tumors.

### SUMMARY OF THE INVENTION

Embodiments incorporating features of the present invention comprise low-cost devices and methods for simultaneous emission and transmission scan during PET scanning. In some embodiments incorporating features of the present invention, one or more detectors are configured with a radioactive material, such as a tube or rod comprising a radioactive material. These detectors generate trigger signals upon impact by short-range radiation emitted by the radioactive material in the tube or rod.

Short-range radiation is radiation that has a range between a micro-meter to one centimeter in water, such as electrons, positrons, auger electrons, and soft x-rays that are emitted from various radio-isotopes. These short range radiations are incapable of penetrating the patient's body and contacting the PET detectors on the other side of the body.

The radioactive material in this detector-rod also emits at least one long range gamma ray substantially simultaneously with the said short range radiation.

A long range gamma ray is a gamma ray with energy between 59 keV and 800 keV. These long range gamma rays are capable of penetrating the patient's body and contacting the PET detectors on the other side of the body.

The attenuation map obtained with these long range gamma rays will be adjusted, using standard mathematical methods, to generate the attenuation map of the patient's body as if 511 keV gamma rays had passed through the body. This attenuation map is needed for correcting the emission PET scans, since the emission PET scan is formed by 511 keV gamma rays emitted from the radioactivity in the patient's body.

A feature of the embodiments described herein is that the detectors in the rod are relatively insensitive to detecting the long range gamma rays. This can be achieved by using low density and low atomic number detector materials in forming the rod detectors. The thickness of detector material must be kept low to minimize the detection of long range gamma rays in the rod detector.

If the rod detectors are made smaller than 1 millimeter and the PET detectors are also similarly small in size, then the attenuation scans made with the long range gamma rays may have diagnostic values similar to x-ray computed tomograms.

In one embodiment incorporating features of the present invention, a Positron Emission Tomography (PET) device comprises multiple scan detectors configured to detect radiation emitted from a first radioactive material having been previously placed into a patient's body, said patient's body placed within the PET device, and at least one radioactivity source within a detector structure positioned within the PET device and spaced from the first PET scanner detectors. The detector structure comprises at least one radioactivity source, which is a second radioactive material, and at least one radiation detector, wherein the at least one radiation detector is configured to detect one or more short-range emissions emitted from the second radioactive material and to generate a signal in response to the detected emissions from the second radioactive material.

In another embodiment incorporating features of the present invention, a device for collecting data during a diagnostic procedure using a Positron Emission Tomography (PET) device comprises a radioactive material, at least one detector positioned adjacent to the radioactive material, with the at least one detector configured to detect short range radiation, and to generate a signal in response to said detected short range radiation.

In another embodiment incorporating features of the present invention, a device for collecting data during a diagnostic procedure using a Positron Emission Tomography (PET) device comprises a radioactive material, at least one detector positioned adjacent to the radioactive material, with the at least one detector configured to detect short range radiation, emitted from the radioactive material and to generate a signal in response to said detected short range radiation.

In yet another embodiment incorporating features of the present invention, a method of correcting attenuation in a Positron Emission Tomography (PET) scan comprises the steps of generating a first set of input data from a radioactive rod detector configured with a radioactive material, with the first set of input data comprising the location and time of an emission event captured in the radioactive rod detector, simultaneously or substantially simultaneously generating a second set of input data from PET detectors located on the internal wall of a PET scanner, with the second set of input data comprising the location and time of the interaction of a long range gamma ray with PET scanner's detector, comparing the first set of input data with the second set of input data, and determining whether to use said second set of data as a transmission event in correcting the PET scan for attenuation of gamma rays by the patient's body.

These and other further features and advantages of the invention are apparent to those skilled in the art from the following detailed description, taken together with the accompanying drawings, wherein like numerals designate corresponding parts in the figures, in which:

### BRIEF DESCRIPTION OF THE DRAWINGS

FIG. 1 is a schematic view of a conventional art PET scanning device;

FIG. 2A is a schematic view of a PET scanning device with a positron emitting isotope inside the rod incorporating features of the present invention;

FIG. 2B is a schematic view of a PET scanning device with a non-positron emitting isotope inside the rod incorporating features of the present invention;

FIG. 2C is a schematic view of a portion of a PET scanning device and rod detector incorporating features of the present invention;

FIG. 2D is a schematic view of a portion of a PET scanning device and rod detector incorporating features of the present invention;

FIG. 2E is a schematic view of a portion of a PET scanning device and rod detector incorporating features of the present invention;

FIG. 3A is side view of a detector arrangement incorporating features of the present invention;

FIG. 3B is side view of another detector arrangement incorporating features of the present invention;

FIG. 4 is a bottom view of an embodiment of detector arrangement as shown in FIG. 3;

FIG. 5 is side view of another detector arrangement incorporating features of the present invention;

FIG. 6 is a sectional side view of another detector arrangement incorporating features of the present invention;

FIG. 7 is a sectional front view of the detector arrangement of FIG. 6;

FIG. 8 is a side view of the detector arrangement of FIG. 6;

FIG. 9 is a sectional side view of a detector arrangement incorporating features of the present invention;

FIG. 10 is a sectional front view of the detector arrangement of FIG. 9;

FIG. 11 is a side view of the detector arrangement of FIG. 9;

FIG. 12 is a schematic view of a PET scanning device incorporating features of the present invention;

FIG. 13 is a decay scheme diagram depicting the decay of Iodine-131 and Cesium-131.

FIG. 14 is a schematic diagram of a method of device trigger control incorporating features of the present invention;

FIG. 15 is a schematic diagram of another method of device trigger control incorporating features of the present invention; and

FIG. 16 is a schematic diagram of another method of device trigger control incorporating features of the present invention.

### DETAILED DESCRIPTION OF THE INVENTION

Embodiments incorporating features of the present invention provide a low-cost mechanism for simultaneous emission and transmission scan during PET scanning. This process improves upon attenuation correction and is also less noisy than other techniques. In some embodiments incorporating features of the present invention, one or more detectors are configured with a radioactive material in the device, such as with a tube or rod comprising a radioactive material. These detectors generate trigger signals upon impact by positrons or other short-range radioactive emissions. Such positrons subsequently decay into two gamma rays and trigger signals enabling differentiation of the resulting annihilation photons from those emitted from the patient's body. The detectors configured with the radioactive material can produce an input of detection values simultaneously with values generated by a surrounding PET scanner detector.

Since embodiments incorporating features of the present invention allow for a transmission scan during the emission scan, the same gating signal can be utilized for coordinating the transmission data into different frames according to cardiac or respiration cycle. While the PET scanner utilizes the rotating source, it can also benefit from gating the transmission scan. By gating the transmission scan, an attenuation map can be accurately matched with the “gated” emission scan, thus reducing the artifacts caused by attenuation mismatch.

In the description that follows, numerous details are set forth in order to provide a thorough understanding of the invention. It will be appreciated by those skilled in the art that variations of these specific details are possible while still achieving the results of the invention. Well-known elements and processing steps are not described in detail in order to avoid unnecessarily obscuring of the description of the invention.

Embodiments of the invention are described herein with reference to illustrations that are schematic illustrations of embodiments of the invention. As such, the actual size, components and features can be different, and variations from the shapes of the illustrations as a result, for example, of technological capabilities, manufacturing techniques and/or tolerances are expected. Embodiments of the invention should not be construed as limited to the particular shapes or components of the regions illustrated herein but are to include deviations in shapes/components that result, for example, from manufacturing, or technological availability, for example, the PET detector ring does not necessarily have to be circular. The regions illustrated in the figures are schematic in nature and their shapes are not intended to illustrate the precise shape or functionality of a feature of a device and are not intended to limit the scope of the invention. Also, components may be shown as one unit but may instead be a collection of components or units, or a collection of components or units may exist as one unit.

Throughout this description, the preferred embodiment and examples illustrated should be considered as exemplars, rather than as limitations on the present invention. As used herein, the term “invention,” “device,” “method,” “present invention,” “present device” or “present method” refers to any one of the embodiments of the invention described herein, and any equivalents. Furthermore, reference to various feature(s) of the “invention,” “device,” “method,” “present invention,” “present device” or “present method” throughout this document does not mean that all claimed embodiments or methods must include the referenced feature(s).

It is also understood that when an element or feature is referred to as being “on” or “adjacent” to another element or feature, it can be directly on or adjacent the other element or feature or intervening elements or features may also be present. It is also understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

Relative terms such as “outer”, “above”, “lower”, “below”, “horizontal,” “vertical” and similar terms, may be used herein to describe a relationship of one feature to another. It is understood that these terms are intended to encompass different orientations in addition to the orientation depicted in the figures.

Although the terms first, second, etc. may be used herein to describe various elements or components, these elements or components should not be limited by these terms. These terms are only used to distinguish one element or component from another element or component. Thus, a first element or component discussed below could be termed a second element or component without departing from the teachings of the present invention. As used herein, the term “and/or” includes any and all combinations of one or more of the associated list items.

The terminology used herein is for describing particular embodiments only and is not intended to be limiting of the invention. As used herein, the singular forms “a,” “an,” and “the” are intended to include the plural forms as well, unless the context clearly indicates otherwise. It will be further understood that the terms “comprises,” “comprising,” when used herein, specify the presence of stated features, integers, steps, operations, elements, and/or components, but do not preclude the presence or addition of one or more other features, integers, steps, operations, elements, components, and/or groups thereof.

It is understood that when the present disclosure references a “tube” or “rod” containing a radioactive material, the “tube” or “rod” need not be conventional shapes and dimensions commonly thought of as “tube-shaped” or “rod-shaped”, for example, a cylinder; although such shapes are acceptable. Instead, the device component containing the radioactive material, referred to as a “tube” or “rod” can be any feasible shape that can work in conjunction with the device, including, but not limited to, any regular or irregular polygon.

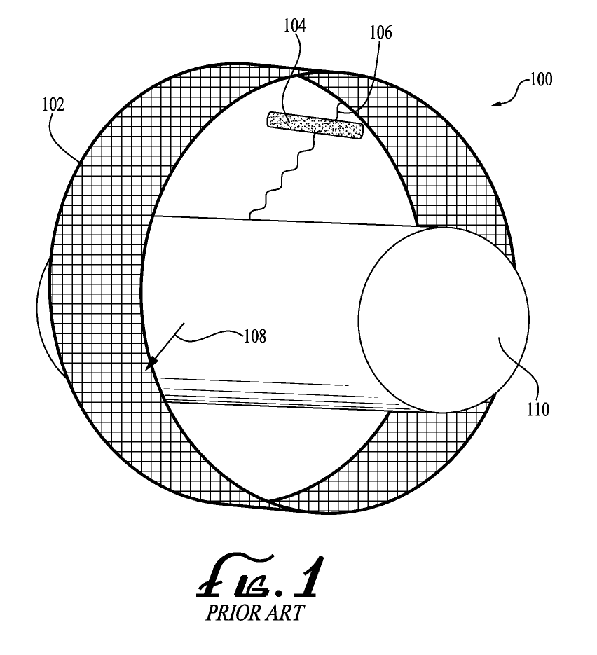

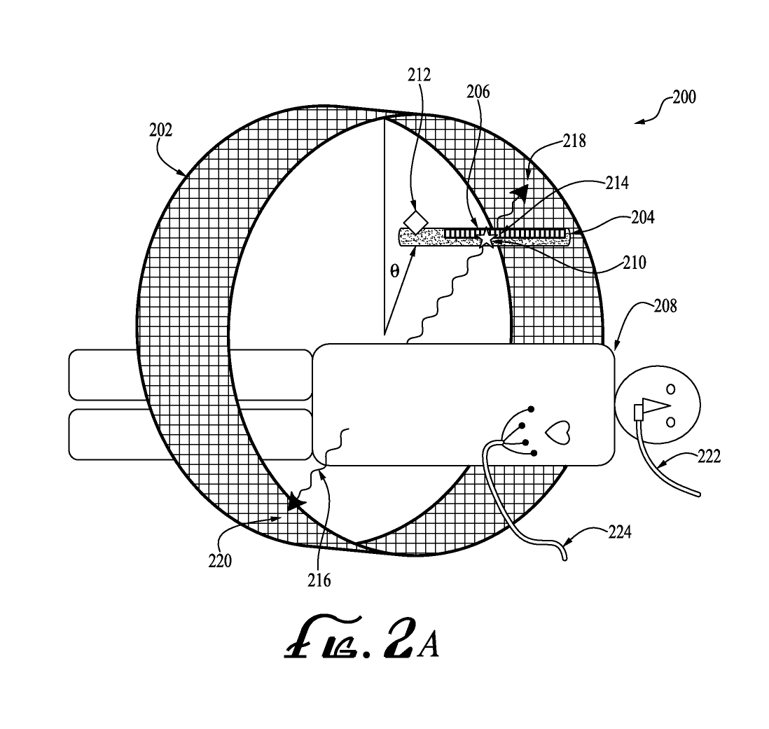

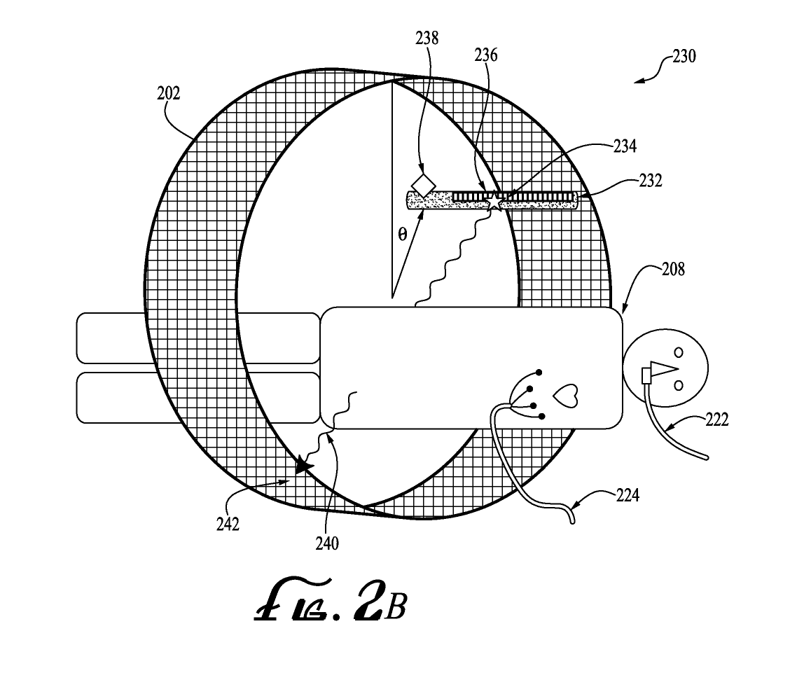

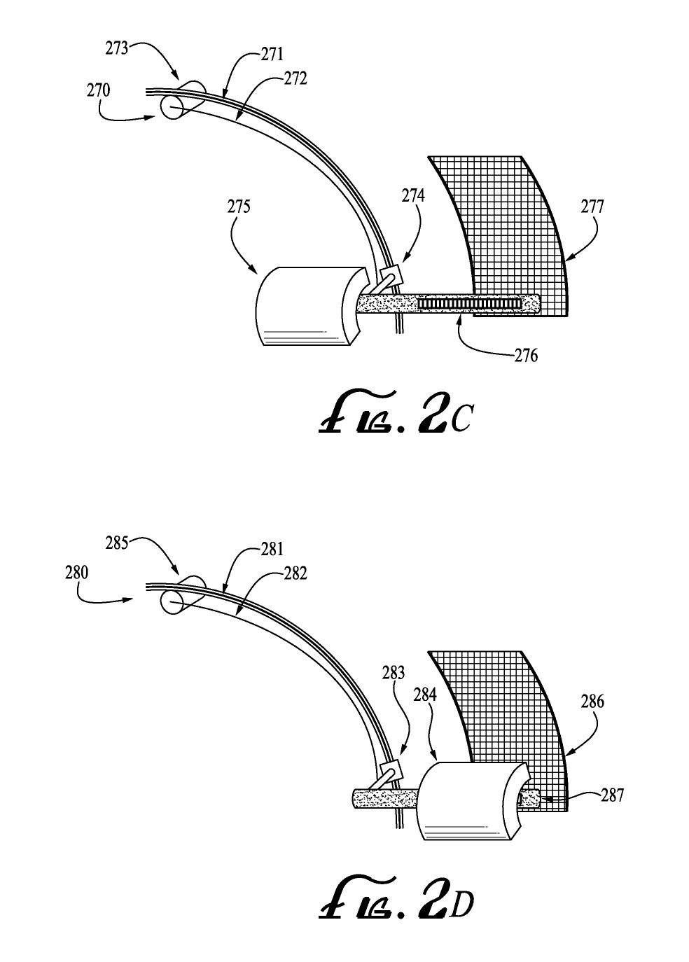

FIG. 2A shows a PET scanner 200, similar to PET scanner 100 in FIG. 1 above, comprising scanning detectors 202 and a positron-emitting radioactive material 204, which can be placed in or otherwise organized into or with a rod or other component-body as shown. In the embodiment of FIG. 2A, the radioactive material is Germanium-68 (GE-68) Unlike, the PET scanner 100 in FIG. 1 above, the PET scanner 200 of FIG. 2 further comprises one or more rod detectors 206 (an array of detectors are shown) configured with (e.g. inside, coupled to or connected to) the positron-emitting rod 204. These rod detectors 206 can be embedded inside the positron-emitting Ge-68 rod 204, coupled or connected to the rod or otherwise arranged or configured with the rod. These rod detectors 206 can be configured to detect a variety of variables, such as particle emissions, including positrons and various types and levels of radiation and radioactivity, for example, alpha, beta, Auger electrons or low energy x-ray radiation.

Shortly after emission from the rod 204, a positron 210 contacts the rod detector 206 while the rotating rod 204 is at the θ position as measured by the angle sensor 212. Two gamma rays, a first gamma ray 214 and a second gamma ray 216, are generated from the positron 210 and hit the PET detectors 202 at a first PET detector position 218 and a second PET detector position 220 respectively. The time-of-travel of the second gamma ray 216 between the rod detector 206 and the second PET detector position 220 is equal to its distance traveled divided by the speed of light.

The distance traveled by the gamma ray 216 is known because the angular coordinate of the rod is measured by the angle sensor 212, the radius is fixed, and the position of the rod detector 206 and the second PET detector position 220 is also known. The time elapsed between a signal from the rod detector 206 and the second PET detector position 220 can be measured directly, within the accuracy of time measurements and the decay time of positron annihilation (124 pico-second). The recorded event at the second PET detector position 220 is a transmission event if these two time durations are equal. If this condition is not satisfied, the PET detector event is either a random event that is discarded (rejected), or it was an emission event emanating from the patient's body 208.

This rejection of random events helps increase the signal to noise of the transmission scan and, in turn, allows gating of the transmission events to different frames according to the respiration or cardiac cycle as measured by a respiration monitor 222 or electrocardiogram monitor 224. It also allows providing more radioactivity from material in the rod, as well as using multiple rods, without overwhelming the PET detector system. The first gamma ray 214 emitted by the annihilation of the positron 210 can also be detected by first PET detector position 218. This further confirms the validity of the transmission event.

Although a Ge-68 containing rod 204 is described above, other positron-emitting isotopes can be utilized. Furthermore, some isotopes that emit no positrons can be utilized. These isotopes preferably: 1) emit a photon with energy more than 59 keV in coincidence with another beta or alpha emissions, or very low energy x-rays (˜10 keV) or Auger electrons, 2) they have long half life isotopes (such as Am-241), or 3) they contain isotopes that have half lives in the order of a few days (such as I-131 with an 8-day half-life) and are readily commercially available.

FIG. 2B shows a PET scanner 230, similar to PET Scanner 200 in FIG. 2A above, wherein like reference numbers denote like features. Like the PET scanner 200 in FIG. 2A above, the PET Scanner 230 in FIG. 2B comprises scanning detectors 202, which can be arranged into a ring form (as shown), in which a patient's body 208 can be placed and the patient's cardiac and respiratory cycles can be measured by a respiration monitor 222 or electrocardiogram monitor 224. Unlike the PET scanner 200 in FIG. 2A above, the PET scanner 230 in FIG. 2B comprises a non-position-emitting radioactive material 232, which can be placed in or otherwise organized into or with a rod or other component-body as shown.

Shortly after the decay of the radioactive isotope 234, its emission, either a beta, alpha, Auger electron, or x-ray depending on various factors such as the isotope chosen, hit the rod detector 236 while the rotating rod is at the θ position as measured by the angle sensor 238. As above, the rod detectors 236 can be embedded in the rod 232 or otherwise be configured with, integrated into or connected to the rod 232. At least one gamma ray 240 is also emitted substantially simultaneously from the non-positron-emitting radioactive material 232. The gamma ray 240 can contact the scanning detector 202 at an event-detecting position 242.

The time-of-travel of the gamma ray 240 between the rod detector 236 and event-detecting position 242 is equal to its distance traveled divided by the speed of light. As above, this distance is known because the angular coordinate of the rod is being measured by the angle sensor 238, the radius is fixed, and the position of the rod detector 236 and the event-detecting position 242 are also known.

The time elapsed between the rod detector 236 and event-detecting position 242 can be measured directly, within the accuracy of time measurements and the decay scheme of the radioisotope. As above, the recorded event in the event-detecting position 242 is a transmission event if these two time durations are equal. If this condition is not satisfied, the PET detector event is either a random event that will be discarded (rejected), or it was an emission event emanating from the patient's body 223.

FIG. 2C shows an example of rod detector arrangement 270 incorporating features of the present invention. The rod detector is attached to a rail 271 and is moved by a motion-transfer cable 272 that is in turn connected to an electro-motor 273. At the point of connection to the rail there is an optical encoder 274 acting as the angle sensor. Alternatively, other methods of position sensing may be used if appropriate.

In order to reduce the radiation dose to the workers that may come close to the PET scanner while it is not in use, a shell or shield 275 can be attached to the rod such that it can cover the radioactive portion of the rod. This shell or shield 275 can be made of a variety of materials, including materials resistant to or absorptive to various forms or radiation, such as lead or tungsten, and can comprise a variety of suitable shapes, for example any regular or irregular polygon shapes. In the embodiment shown, the shield 275 is a cylindrical shape and is made of tungsten. This shield 275 can be retracted when the PET scanner is in use. Tungsten is preferably used because of its high absorption of the gamma rays. The thickness of this shield can vary and in some embodiments is between 3 mm and 30 mm.

FIG. 2D shows an alternative rod detector arrangement 280, showing the shield 284 covering the radioactive source embedded in the rod 287. FIG. 2D further shows the rod 287 attached to a rail 281 and is moved by a motion-transfer cable 282, that is in turn connected to an electro-motor 283. At the point of connection to the rail there is an optical encoder 283 acting as the angle sensor.

FIG. 2E shows another embodiment of this shield formed as a semi-cylinder 297. In this embodiment, the shield 297 is rotated to cover or uncover the radioactive source 296 emitting gamma rays toward the center of the PET scanner 295. In this configuration, the shield 297 would go between PET scanners 295 that are near the rod 296 and the radioactivity in the rod. This has a salutary effect of reducing the number of unwanted gamma rays that may hit the near detectors 295. FIG. 2E further shows the rod 296 connected to a rail 292 and moved by a motion-transfer cable 293, that is in turn connected to an electro-motor 294. At the point of connection to the rail there is an optical encoder 294 acting as the angle sensor.

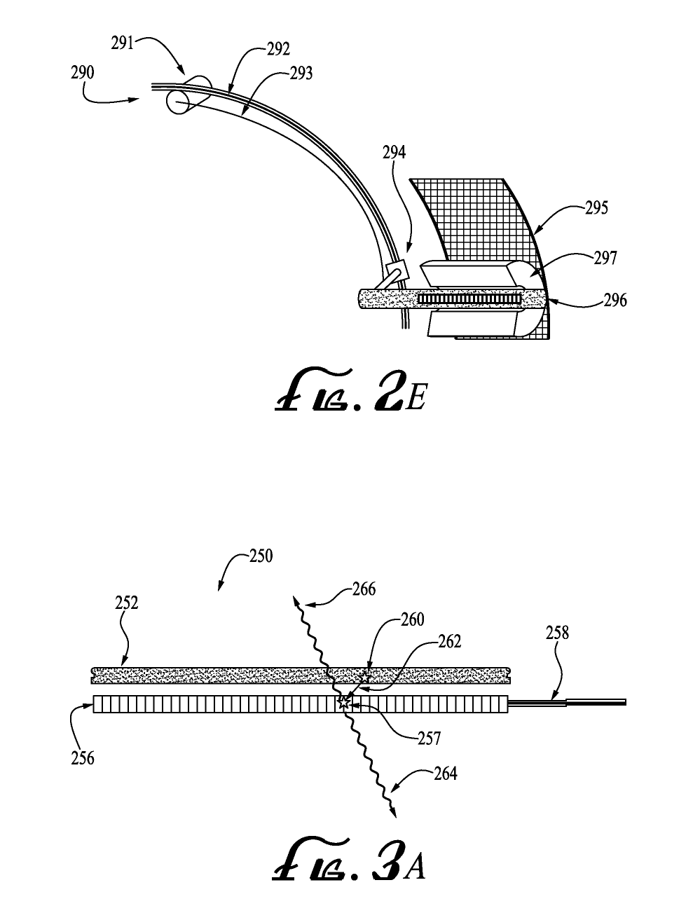

FIG. 3A shows an example of a rod detector arrangement 250 incorporating features of the present invention. FIG. 3 shows a positron-emitting radioactive material 252, which is similar to the Ge-68 containing rod 204 in FIG. 2A above. FIG. 3A further shows one or more rod detectors 234 (an array 256 of rod detectors is shown). FIG. 3A further shows a carrying cable 258 arranged to carry signals to and from the rod detector array 256. Any suitable cable, wired or wireless based transmission arrangement known in the art can be utilized for this purpose.

FIG. 3A further shows radioactive decay 260 occurring, resulting in a positron emission 262, being emitted from the radioactive material 252 and absorbed by the one or more rod detectors 254 in the rod detector array 256. This positron emission 262 is annihilated with an electron, producing a first gamma ray 264 and a second gamma ray 266. The time delay between positron emission and its annihilation into two gamma rays has a short decay time of approximately 124 picoseconds. One of the gamma rays 264, 266 passes through a patient and is detected by the surrounding PET scanning detectors (not shown), forming a transmission scan.

FIG. 3B shows an example of a rod detector arrangement 300 incorporating features of the present invention incorporating a non-positron emitting radioactive material 302. FIG. 3B further shows one or more rod detectors 304 (an array 306 of rod detectors is shown). FIG. 3B further shows a carrying cable 308 arranged to carry signals to and from the rod detector array 306. Any suitable cable, wired or wireless based transmission arrangement known in the art can be utilized for this purpose.

FIG. 3B further shows radioactive decay 310 occurring, resulting in a first radiation emission 312, for example, alpha, beta, Auger electrons, or low energy x-ray radiation, being emitted and absorbed by the one or more rod detectors 304 in the rod detector array 306. The radioactive decay 310 further results in a gamma ray 314 emitted by the radioactive source 302 substantially simultaneously with the first radiation emission 312. The gamma ray 314 passes through a patient and is detected by the surrounding PET scanning detectors (not shown), forming a transmission scan.

In one embodiment incorporating features of the present invention, alpha emission is used for triggering. With continued reference to FIG. 3B, the radioactive material 302 in this embodiment can comprise Americium-241 (Am-241), which is placed on top of one or more alpha detectors 304 (e.g. surface barrier semiconductor detectors) that are placed next to each other making at least one row 306. The radioactive Am-241 302 emits alpha particles 312 in coincidence with gamma rays 314 in the dominant decay scheme of (86%), the Am-241, emitting an alpha particle with energy of 5.5 MeV and transforming to an exited state of Neptunium-237 (Np-237), which in turn emits a gamma ray of 59.5 keV energy with a half-life of 65 ns, the gamma rays 312 constitute the transmission photon. The 59.5 keV gamma rays can traverse a patient's body and be detected in the PET scanners detectors (not shown), and can further be utilized with a coincidence time and/or energy window for filtering the transmission data from the emission data. The energy window can also be used to further discriminate against 511 keV emission data when forming the transmission data set.

Many different types of radiation can be utilized with embodiments incorporating features of the present invention. In another embodiment, beta particles are used as trigger emissions. With continued reference to FIG. 3B, in this beta particle embodiment, Iodine-131 (I-131) is utilized as the radioactive material 302 and is placed on an array of beta detectors 306. Suitable beta detectors include Silicon photomultipliers (SiPMs), which can be coupled or otherwise configured with scintillators, for example, plastic scintillators. The gamma rays 314, typically around 364 keV, are used for the transmission scan and are emitted simultaneously with the beta rays 312.

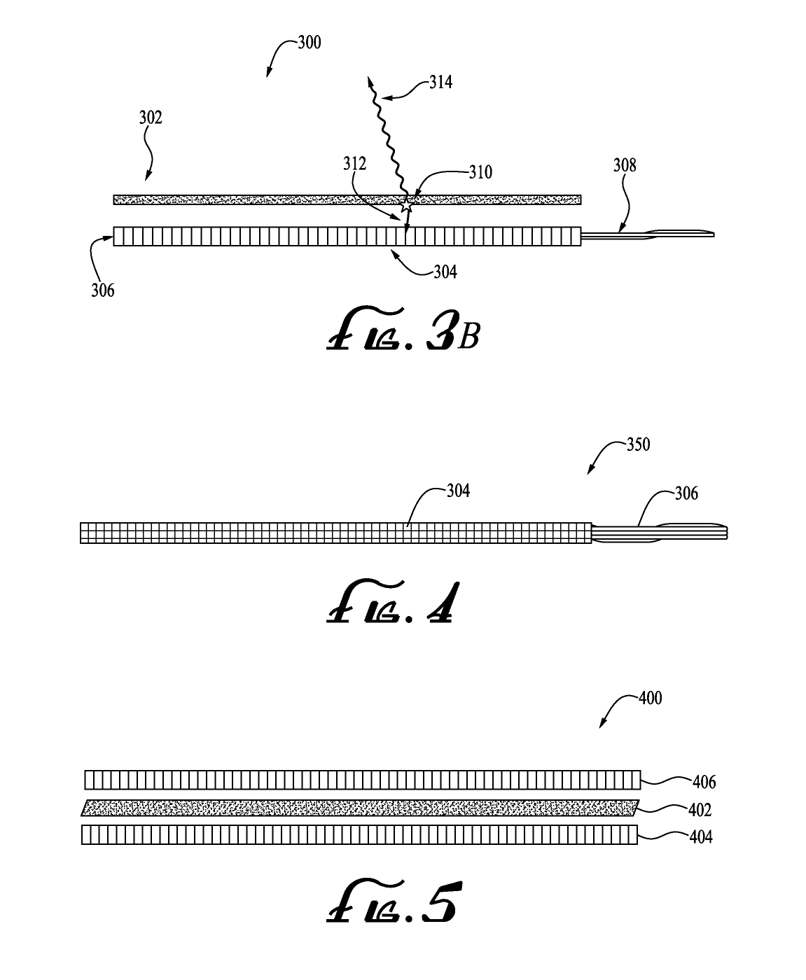

In yet another embodiment, with continued reference to FIG. 3B, soft x-rays 312 (approximately under 30 keV energy) are used as the trigger emissions. These soft x-rays are emitted simultaneous with higher energy gamma rays 314. In these embodiments, an acceptable radioactive material is cobalt-57 (Co-57), which emits gamma rays of approximately 122 keV in coincidence with soft x-rays. The detector array 306 that is in contact with the radioactive element 302 may be built by coupling thin layers of scintillator (such a Brilliance® by Saint Gobain S.A. to SiPMs). A bottom view of such a rod detector configuration 300 is shown in FIG. 4, which depicts the rod detector arrangement 304 and the signal cable 326 of FIG. 3B.

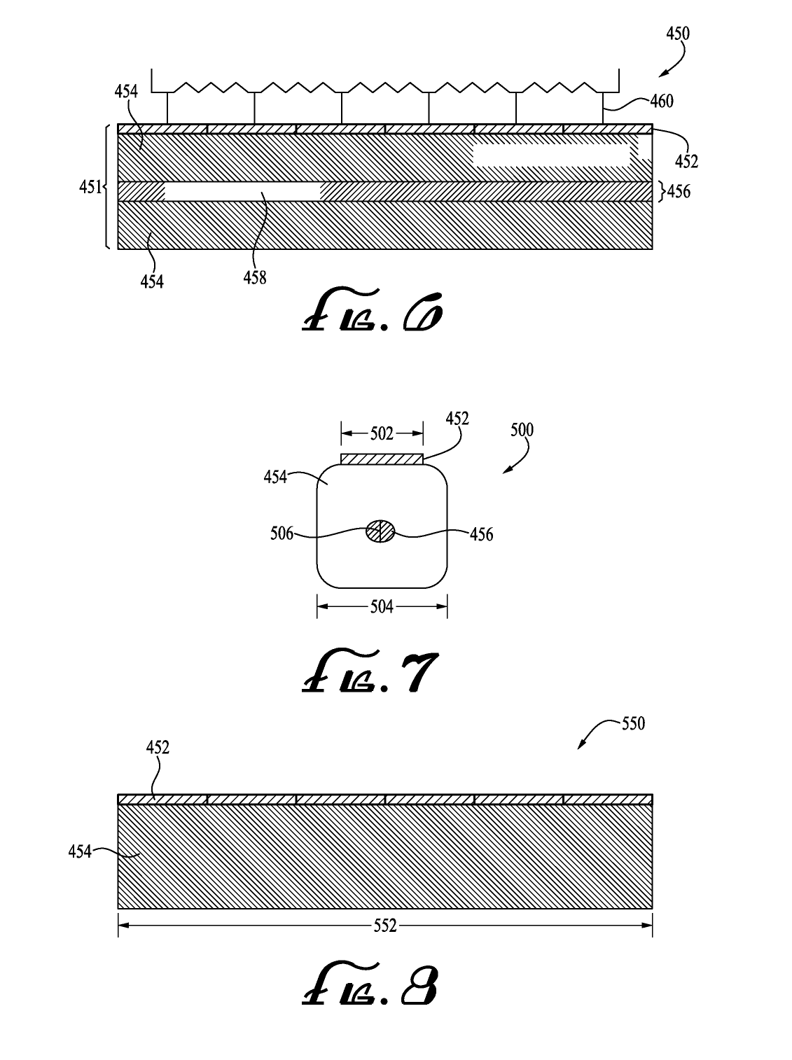

While the embodiments referencing FIGS. 3A and 3B above show examples of positioning of the radioactive material 252, 302 in relation to the rod detectors 256, 306 it is understood that many different additional arrangements are possible. FIG. 5 shows a detection configuration 400, comprising a radioactive source 402, similar to the radioactive source 252 in FIG. 3A above, between a first a array of rod detectors 404 and a second array of rod detectors 406. Any of the above-described radioactive material and radiation-type embodiments can be configured in this manner. This FIG. 4 increases the number of trigger emissions that can be detected in two dimensions therefore reducing the probability of missing those emissions.

Two further examples of rod detector arrangements incorporating features of the present invention are shown in FIGS. 6-8 and 9-11, which show various examples of SiPMs utilized with the rod detectors. FIGS. 6-8 show one embodiment of a rod detector arrangement 450, comprising one or more SiPMs 452 coupled to the rod 451 and including one or more scintillators 454 (which can be made of any suitable material, for example, plastic scintillator), and can be any suitable shape, such as the rectangular shape shown. There is a channel 456, which can be cylindrical, and which is formed throughout the length of this rod.

This channel 456 can be completely internal to the device (as shown), at least partially internal, or can be otherwise connected or coupled to a portion of the device. A radioactive material 458, such as any of the radioactive materials discussed above with reference to FIGS. 3A and 3B, is deposited in this channel 456 at the center of this rod 451. In the embodiment shown the radioactive material is Sodium-22 (Na-22). Transmission components 460, such as are be coupled to SiPMs 452, allowing signals to be transmitted to and from the detector 450. The ideal length of the rod would be determined according to technical feasibility. For example, if the rod can be made 18 millimeters (mm) long, then several SiPM-based cameras would be used to cover the entire 220 mm of the axial dimension. It is understood that the various rod detector dimensions, and one-dimensional as well as two-dimensional arrays of detectors, disclosed herein refer to specific embodiments and other dimensions can be utilized with the devices disclosed herein.

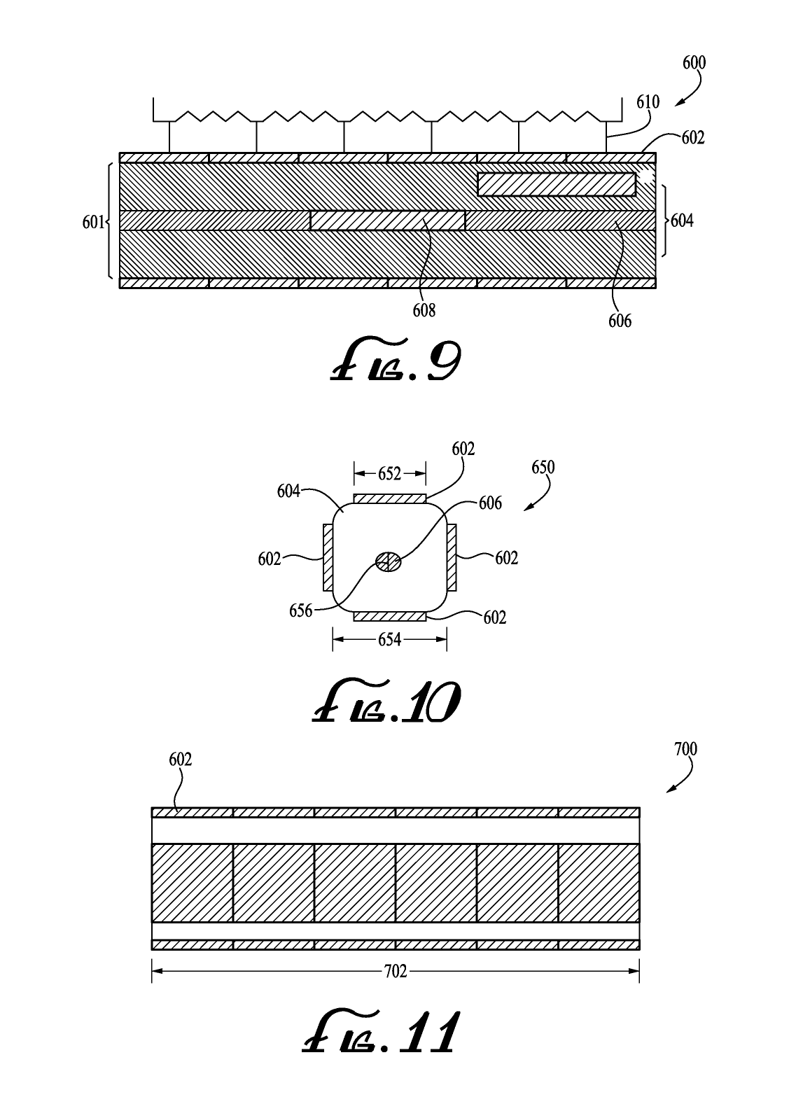

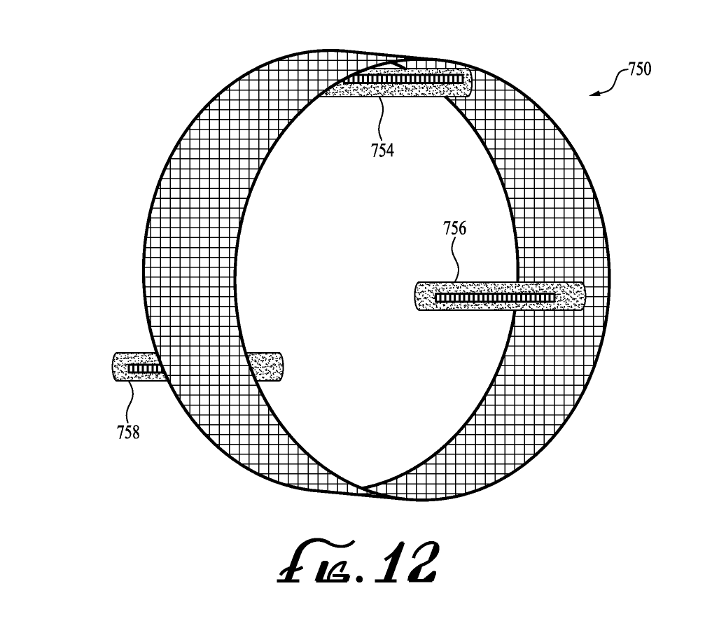

FIG. 7 shows a front cross-sectional view 500 of the rod detector arrangement 450 in FIG. 6 above. As above, the rod detector arrangement 500 comprises one or more SiPMs 452, a scintillator 454 and a channel 456. FIG. 7 also shows some example dimensions for these various components. The SiPMs 452 can have a width 502 of approximately 3 mm with the width 504 of the scintillator portion 454 being approximately 4 mm. The channel 456, in this case cylindrical in shape, can have a diameter 506 of approximately 0.5 mm. FIG. 8 shows further dimensions of the rod detector arrangement 450 in a side view 550. This side view 550 shows the SiPMs 452 and the outer portion of the scintillator 454. This view also shows the rod length 552 dimension, which in this embodiment is approximately 18 mm. In other embodiments, the rod can have a rod length of between approximately 1 mm and approximately 220 mm, although as mentioned above the various rod detector dimensions disclosed herein refer to specific embodiments and other dimensions can be utilized with the devices disclosed herein.

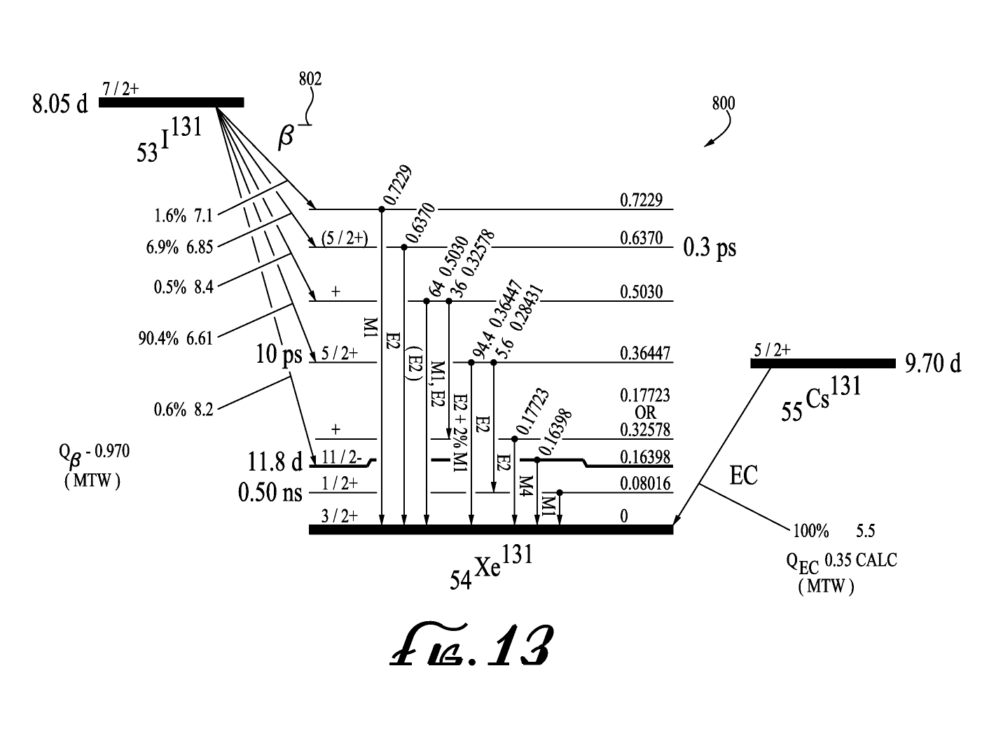

FIGS. 9-11 show another rod detector arrangement embodiment, similar to the rod detector arrangement 450 shown in FIG. 6 above, but different in that the number of SiPMs are increased to improve detection. FIGS. 9-11 show one embodiment of a rod detector arrangement 600, comprising SiPMs 602 on each side of the device. The device shown in FIG. 9 is rectangular with all four sides of the device having a SiPM array as shown in more detail in FIG. 10. The SiPM arrays are coupled to the rod 601 which comprises a scintillator 604 (which, as above, can be made of any suitable material, for example, a plastic scintillator). There is a channel 606, which can be cylindrical, shown as extending along the length of the rod 601. As above, this channel 606 can be completely internal to the device (as shown), at least partially internal, or can be otherwise connected or coupled to a portion of the device.

A radioactive material 608, such as any of the radioactive materials discussed above with reference to FIG. 3A or 3B is deposited in this channel 606 at the center of this rod 601. Transmission components 610, such as wires, cable, or wireless transmission devices, can be coupled to the SiPMs 602, allowing signals to be transmitted to and from the detector 600.

FIG. 10 shows a front cross-sectional view 650 of the rod detector arrangement 600 in FIG. 9 above. The rod detector arrangement 600 comprises SiPMs 602, a scintillator 604 and a channel 606. The device of FIG. 10 can have the same dimensions as the device of FIG. 7 above, including the SiPMs 602 with a width 652 of approximately 3 mm and a width 654 of the scintillator portion 604 being approximately 4 mm. The channel 606, shown as cylindrical in shape, can have a diameter 656 of approximately 0.5 mm. FIG. 11 shows further dimensions of the rod detector arrangement 600 in a side view 700. This side view 700 shows the SiPMs 602 on all sides of the device and the outer portion of the scintillator 604. This view also shows the rod length 702 dimension, which in this embodiment is approximately 18 mm.

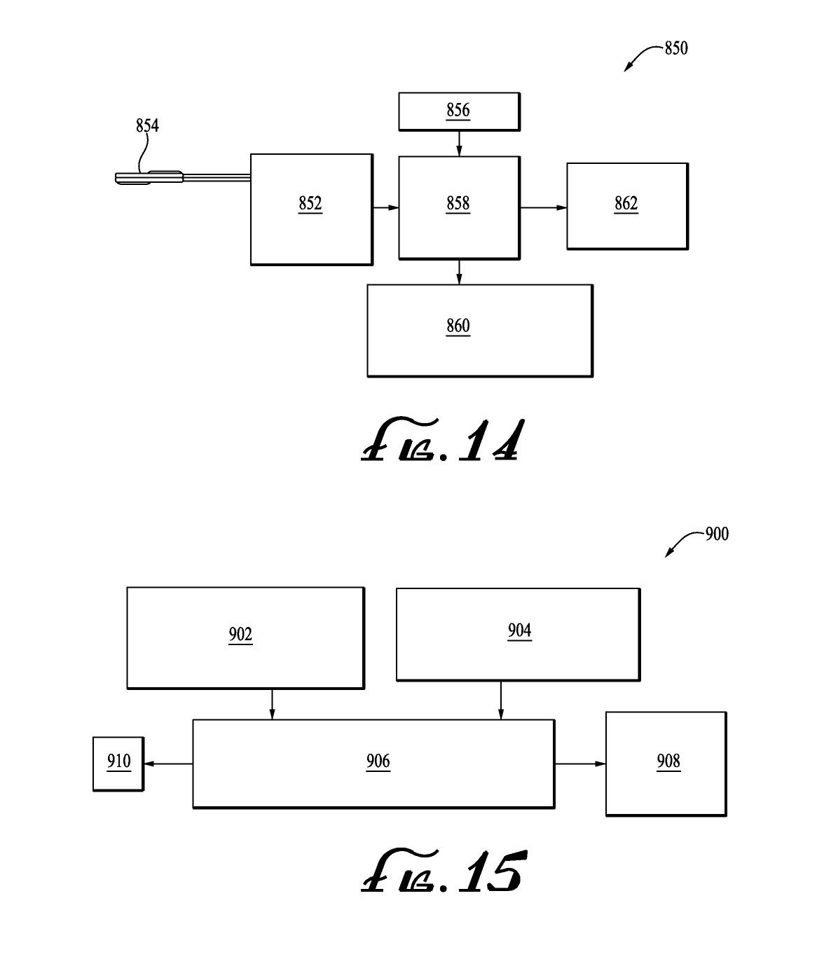

PET scanners including multiple rods, and accordingly, multiple rod detectors, are also within the scope of embodiments incorporating features of the present invention. FIG. 12 shows an embodiment of a PET scanner device 750, comprising scanner detectors 752, a first rod 754, a second rod 756 and a third rod 758. First, second and third rods 754, 756, 758 contain both a radioactive material and rod detectors as in embodiments above. One advantage of utilizing a multiple-rod embodiment is that detection can be performed at a greater rate, with the various rods dedicated to different portions of the PET scanner device 750. Rather than having a single rod rotate about the entire perimeter, multiple rods can be utilized simultaneously, each rod moving along a certain pre-determined path.

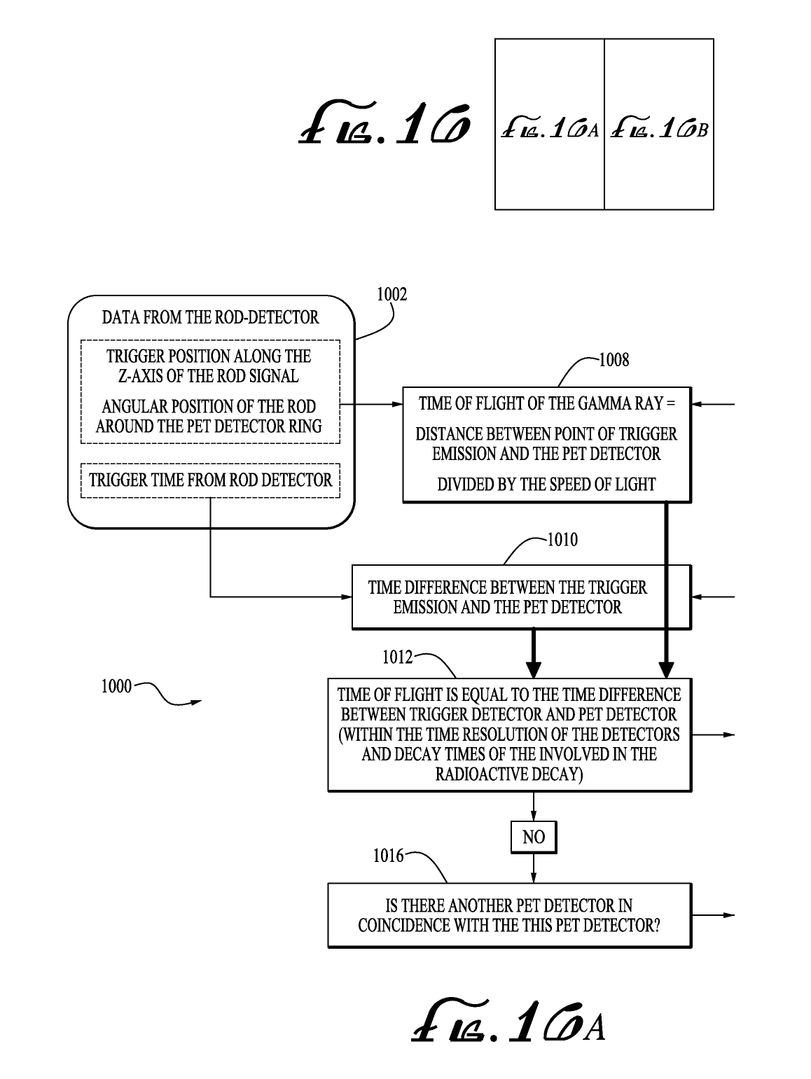

It is important to understand the decay properties of the radioactive material selected. FIG. 13 shows an example decay scheme 800 for the embodiment mentioned above utilizing I-131. As shown in decay scheme 800, I-131 decays into Xenon-131, releasing beta radiation 802. As mentioned above, the decay rate corresponds to roughly an 8-day half-life. Suitable beta detectors include Silicon photomultipliers (SiPMs) and the gamma rays for the transmission scan and are emitted simultaneously with the beta rays.

FIG. 14 shows an example method of use 850 of the PET scanner device disclosed herein. A first input step 852 includes receiving data from rod detectors, for example, time trigger and information regarding the position of the radioactive material rod. This data can be received from a transmission component 854, for example, a cable, wire or wireless device connected to or coupled to the radioactive rod. Simultaneously or almost simultaneously with the time of the first input step 852, a second input step 856 is executed, wherein data from the scanner detectors is received.

After data is collected from the first input step 852 and the second input step 856, a determination step 858 is executed. In the determination step, events that are within the time window of the radioactive-rod-detector triggers are collected, selected and organized into categories. Example categories include a first category 860, which corresponds to a collection of the events that were not in the time window of coincidence with radioactive rod triggers, and a second category 862, which corresponds to a collection of the events generated by the photons from the radioactive rod. The collected events, 862 are utilized to correct for attenuation.

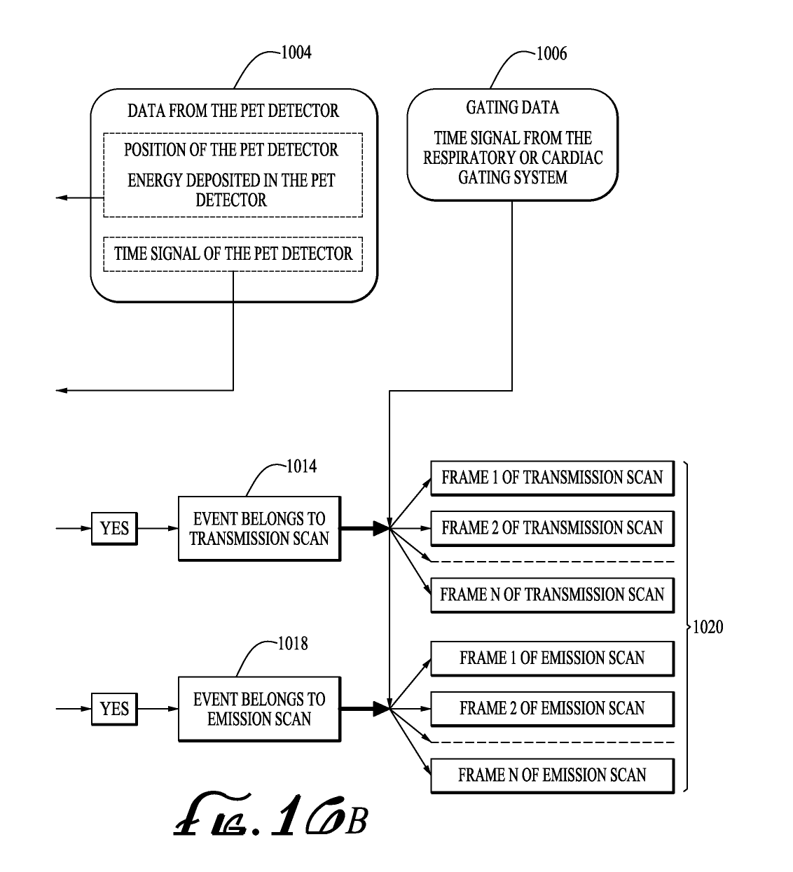

FIG. 15 shows an alternative method of use 900 of devices incorporating features of the present invention utilizing a beta radiation and SiPM camera embodiment. A first input step 902 corresponds to input received by an SiPM detector embedded rod. Example information received in the first input step 902 can include a time trigger from the radioactive rod, positioning data such as the angular position of the rod and the position of the rod detector in the array of detectors, for example, the SiPM. A second input step 904 is executed simultaneously or nearly simultaneously with the first input step 902 and corresponds to data received by the PET scanner. Such data received in the second input step can include the position of the detection in the detector and the time of the event.

After the data are received in the first and second inputs steps 902, 904, the data is collected and a calculation is performed in a calculation step 906. An example calculation utilizing the data from the input steps 902, 904, includes, as an example, determining whether the time of the PET event from the second input step 904 minus the time of the trigger event from the rod detector in the first input step 902 is equivalent to the time traveled by the photon during its travel between the position of the beta detector determined from the first input step 902 and the detector position of the PET scanner determined from the second input step 904. If these values are in fact equivalent, one can move on to a positive determination step 908 and the PET event is then utilized as the transmission event. If the above values are not equivalent, then one can move on to a negative determination step 910 and the process can be repeated until a positive determination can be made.

It is understood that the method set forth above with regard to FIG. 15 utilizing SiPM detectors and beta radiation can likewise be applied mutatis mutandis to any of the other mentioned radioactive material and detector schemes disclosed.

FIG. 16 shows another example method of use 1000, comprising multiple data steps, including data gathering from the rod detector 1002, which can include trigger position along the z-axis of the rod signal, angular position of the rod around the PET detector ring and trigger time from the rod detector. The method 1000 can further comprise the data step of obtaining data from the PET detector ring 1004, which can include information regarding the position of the PET detector, the energy deposited in the PET detector and the time signal of the PET detector. Another data collection step can also be utilized wherein physiologic gating data is collected 1006, which can include one or more time signals from respiratory or cardiac gating system.

Once data from the rod detector 1002 and data from the PET detector 1004 are collected calculation steps can be performed. In a first calculation step 1008, the time of flight of the gamma ray can be calculated by dividing the distance between the point of trigger emission and the PET detector by the speed of light. In a second calculation step 1010, which can occur prior to, simultaneous with or after the first calculation step 1008, the time difference between the trigger emission and the PET detector is calculated.

In a comparison step 1012, it is determined whether the time of the flight of the gamma ray is equal to the time difference between the trigger detector and the pet detector. If the answer is yes, then the event is utilized in the transmission scan 1014. If the answer is no, a second determination step 1016 is performed, wherein it is determined whether there is another PET detector in coincidence with the present PET detector. If the answer is yes, then the event is added to in the emission scan 1018. The gating data 1006 is used with the transmission scan data 1014 and the emission scan data 1018 to divide these two set of data to various frames according to the physiologic cycle 1020.

Although the present invention has been described in detail with reference to certain preferred configurations thereof, other versions are possible. Embodiments of the present invention can comprise any combination of compatible features shown in the various figures, and these embodiments should not be limited to those expressly illustrated and discussed. Therefore, the spirit and scope of the invention should not be limited to the versions described above.

The foregoing is intended to cover all modifications and alternative constructions falling within the spirit and scope of the invention as expressed in the appended claims, wherein no portion of the disclosure is intended, expressly or implicitly, to be dedicated to the public domain if not set forth in the claims.

We claim:

1. A Positron Emission Tomography (PET) device comprising
multiple scan detectors configured to detect radiation emitted from a patient's body placed within the PET device, a first radioactive material having been previously placed into said body; and
at least one emission detector structure positioned within the PET device and spaced from the first scan detectors,
the at least one emission detection structure comprising a second radioactive material and at least one emission detector, said at least one emission detector configured to detect one or more short range radiation emitted from the second radioactive material and to generate a signal in response to said detected emissions from said second radioactive material, said second radioactive material emitting at least a gamma ray substantially simultaneous with at least one short range emission.

2. The device of claim 1, wherein said second radioactive material comprises Germanium-68.

3. The device of claim 1, wherein said second radioactive material comprises Americium-241.

4. The device of claim 1, wherein said second radioactive material comprises Iodine-131.

5. The device of claim 1, wherein said second radioactive material comprises Cobalt-57.

6. The device of claim 1, wherein said emission detector is configured to detect positron emissions.

7. The device of claim 1, wherein said emission detector is configured to detect beta radiation.

8. The device of claim 1, wherein said emission detector is configured to detect x-ray radiation.

9. The device of claim 1, wherein said emission detector comprises at least one Silicon photomultiplier (SiPM).

10. The device of claim 1, wherein said signal generated by said emission detector structure is used to locate the position of the emission detector within the PET device.

11. A device for collecting data during a diagnostic procedure using a Positron Emission Tomography (PET) scanner, said device for collecting data comprising:
a radioactive material, and
at least one detector positioned adjacent to said radioactive material, said at least detector configured to detect short-range radiation emitted from the radioactive material and to generate a signal in response to said detected short-range emission.

12. The device of claim 11, wherein said radioactive material is positioned within a channel within a scintillator.

13. The device of claim 12, wherein said scintillator is plastic scintillator.

14. The device of claim 11, wherein said at least one detector is configured to detect an emission event and to generate a signal indicating the position and the time of the emission event.

15. The device of claim 14, wherein said emission event is a positron emission.

16. The device of claim 14, wherein said emission event is an electron radiation emission.

17. The device of claim 11, wherein said at least one detector is configured to detect an emission event and to generate a signal usable to identify the position of the emission detector and the time of the emission event.

18. The device of claim 11, wherein said radioactive material comprises Iodine-131 and said at least one detector comprises at least one Silicon photomultipliers (SiPM).

19. A method of correcting attenuation in a Positron Emission Tomography (PET) scan, comprising the steps of:
generating a first set of input data from a radioactive rod detector configured with a radioactive material, said first set of input data comprising the location and time of an emission event;
simultaneously or substantially simultaneously generating a second set of input data from at least one scan detector located on an internal wall of a PET scanner, said second set of input data comprising the location and time of a gamma ray interaction;
comparing said first set of input data with said second set of input data; and
using said second set of data as a transmission event in correcting the scan for attenuation.

20. The method of claim 19, wherein said comparison of said first set of input data with said second set of input data comprises:
determining whether the difference between
a) the time of the emission event from the second set of input data and
b) the time of the emission event from the first set of input data
is equivalent to the time traveled by a photon during its travel between the position of the rod scanning detector determined from the first set of input data and the PET scanning detector position determined from the second set of input data.

21. The method of claim 19, wherein determining if a gamma ray detected by scan detectors is a transmission gamma ray utilizes the time-of-flight of a gamma ray between the rod detector rod said scan detector.

22. The method of claim 19 wherein a transmission scan is divided into different frames using time signals from a physiologic gating system based on cardiac cycle or respiratory cycle.

23. The device of claim 11 wherein a radiation shield is positioned to block radiation from said radioactive material when the PET scanner is not in use.
